(19) United States
(12) Patent Application Publication
(10) Pub. No.: US 2026/0183471 A1
(43) Pub. Date: Jul. 2, 2026

(54) MEDICAL DEVICE PACKAGING FOR INHIBITING INADVERTENT ACTIVATION

(71) Applicant: Tandem Diabetes Care, Inc., San Diego, CA (US)

(72) Inventor: Vincent Vaucher, St-Sulpice (CH)

(21) Appl. No.: 19/429,148

(22) Filed: Dec. 22, 2025

Related U.S. Application Data
(60) Provisional application No. 63/738,892, filed on Dec. 26, 2024.

(51) Int. Cl. A61M 5/00; A61M 5/142

(52) U.S. Cl. CPC A61M 5/003; A61M 5/14248; A61M 2209/06

(57) ABSTRACT

Embodiments disclosed herein are directed to packaging for medical devices that reduces the likelihood of inadvertent activation of such devices during shipping, storage and handling of the device by the user while in the packaging. Features can be provided on the packaging adjacent an actuation mechanism of the device that reduce the transmission of force on the outside of the packaging onto the actuation mechanism to inhibit the inadvertent actuation of the mechanism.

### PRIORITY CLAIM

The present application claims the benefit of U.S. Provisional Patent Application No. 63/738,892 filed Dec. 26, 2024, which is hereby incorporated herein by reference in its entirety.

### TECHNICAL FIELD

The present disclosure relates generally to ambulatory infusion pumps and, more particularly, to packaging of ambulatory infusion pumps.

### BACKGROUND

Wearable insulin pumps are known for providing a Type I Diabetes Mellitus patient with small doses of short acting insulin continuously (basal rate). The devices also can be used to deliver variable amounts of insulin when a meal is consumed (bolus). The basal insulin rates are usually programmed in a pump by a physician, and one or multiple basal settings may be programmed in the pump based on the patient's needs. The patient may program the amount of insulin for a mealtime bolus directly on the pump. Most pumps also include bolus calculators to help the patient determine the amount of insulin the patient may need at mealtime based on the patient's glucose levels and the amount of carbohydrates the patient may consume. The objective is to control the patient's blood glucose level within a desired range. Some such insulin pumps are coupled to an adhesive patch that permits the pump to be directly adhered to a user's body surface, for example the abdomen, and are referred to as “patch pumps.” In addition, some previously known systems were configured to interface wirelessly with a continuous glucose monitor, which typically also may be disposed on a patch designed to be adhered to the user's body. Other previously known systems employ still further modules designed to monitor user activity and report that activity to a controller associated with the patch pump to titrate the insulin delivery in accordance with the user's activity level.

For patch pumps, a transcutaneous cannula generally needs to be inserted into the skin for delivering medication transcutaneously from an external fluid reservoir associated with the patch pump that is in fluid communication with the cannula. Thus, the cannula needs to be inserted into the skin before delivering the medication. Patch pump systems may include an applicator that can simultaneously insert the cannula into the user's skin and affix an adhesive pad onto the user's skin that holds the patch pump. The applicator will typically have an actuator on an external housing of the applicator that is actuated to cause internal mechanisms within the housing to insert the cannula into the user's skin and release the adhesive pad onto the user's skin. Such applicators are generally single use applicators that can be actuated only a single time. As such, it is important to avoid inadvertent actuation of the applicator.

### SUMMARY

Embodiments disclosed herein are directed to packaging for medical devices that reduces the likelihood of inadvertent activation of such devices during shipping, storage and handling of the device by the user while in the packaging. Features can be provided on the packaging adjacent an actuation mechanism of the device that reduce the transmission of force on the outside of the packaging onto the actuation mechanism to inhibit the inadvertent actuation of the mechanism.

In an embodiment, a package is configured to contain a medical device configured to be actuated by an external actuation mechanism on the medical device. The package can include a generally planar base and a flexible case selectively attachable to the generally planar base to enclose the medical device between the generally planar base and the flexible case. The flexible case can include a body portion generally conforming to a shape of the medical device and a plurality of ridges extending upwardly from the body portion over the external actuation mechanism when the medical device is enclosed between the generally planar base and the flexible case. External ridges reduce the transmission of forces on plastic case onto the actuation mechanism, which reduces the possibility of inadvertent actuation of the medical device while the medical device is contained within the package.

In an embodiment, a medical device system can include a medical device configured to be actuated by an external actuation mechanism on the medical device and a package including a generally planar base and a flexible case selectively attachable to the generally planar base to selectively enclose the medical device between the generally planar base and the flexible case. The flexible case can include a body portion generally conforming to a shape of the medical device and a plurality of ridges extending upwardly from the body portion over the external actuation mechanism when the medical device is enclosed between the generally planar base and the flexible case.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an exemplary medication infusion system according to the disclosure.

FIG. 2 is a diagram showing exemplary attachment zones for the patch pump of FIG. 1 and an external sensor.

FIGS. 3A and 3B are, respectively, a perspective view and an exploded view of the pad and applicator of FIG. 1.

FIGS. 4A-4D depict the applicator of the medical infusion system of FIG. 1 contained in a packaging.

FIG. 5 is a partial view of the packaging of FIGS. 4A-4D.

FIG. 6 depicts an applicator device.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

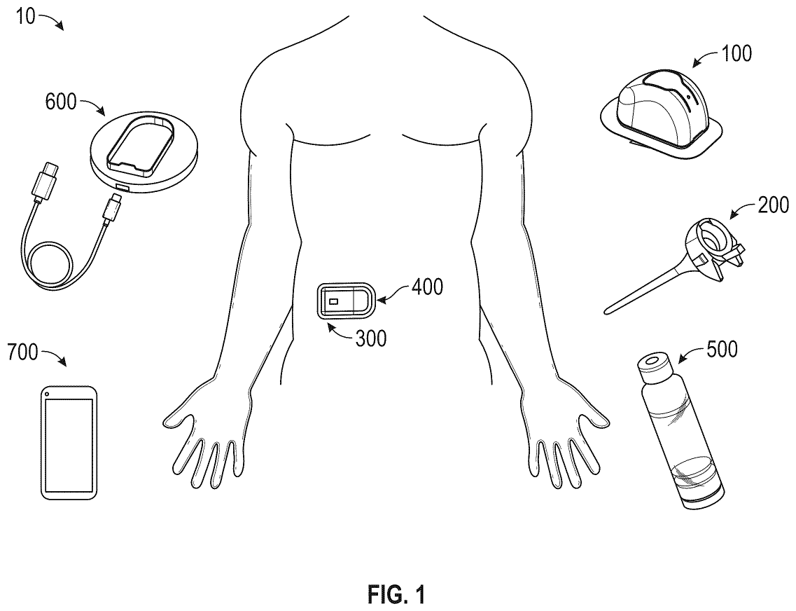

Referring to FIG. 1, an exemplary medication infusion system including a patch pump for delivering medication is described. In FIG. 1, components of the system are not depicted to scale on either a relative or absolute basis. Medication infusion system 10 can include applicator 100, cannula 200, pump 300, cap 400, cartridge 500, charging system 600 and/or software application 700. Preferably, applicator 100, cannula 200, cap 400, and cartridge 500 are disposable components that may be replaced approximately every 3-10 days and/or once the pre-filled cartridge is empty, while pump 300 is reusable and may last for an extended period of time, e.g., approximately 2-4 years. As such, pump 300 may be used with many different applicators, cannulas, caps, and pre-filled cartridges. Such a configuration is expected to promote sanitary use of the system, as the components exposed to the patient and the insulin are disposable, while reducing costs for components containing more expensive electronics, e.g., pump 300, charging system 600, and/or software application 700, which may be used repeatedly. In some embodiments, system 10 includes a second pump, such that the wearer may charge the second pump while using the first pump and vice versa. In this manner, the wearer will always have a pump that is charged and ready to be used once the cartridge of the pump in use is empty. Further, this system can be designed to reduce waste while reducing the number of times the wearer is required to insert a new cannula. Medication infusion system 10 may be used to apply cannula 200 and a pad to a wearer and to deliver medication through cannula 200 via a patch pump coupled to the pad. Further details regarding such a system can be found in U.S. Patent Publication No. 2022/0379014, which is hereby incorporated herein by reference in its entirety.

Applicator 100 is configured to apply an adhesive pad to the wearer and, upon actuation, to insert cannula 200 into the wearer. The pad is configured to be secured to the wearer for a period of time, e.g., at least 3 days, 7-10 days, etc., and then may be replaced by a similar pad using a similar applicator. The pad may include a pad skeleton having one or more locking mechanisms that are configured to couple the pad to applicator 100 for insertion of cannula 200 or to the assembled pump for delivery of medication. Applicator 100 may include an internal component configured to support an insertion mechanism designed to insert cannula 200 through the skin of the wearer via rotational movement and to guide and orient cannula 200 during insertion.

Preferably, applicator 100 is designed to suppress noise during insertion. The insertion mechanism may include an applicator needle configured to pierce the wearer's skin and a biasing member, which may be coupled to one or more links configured to interact with cannula 200 and the applicator needle. Upon actuation by the wearer, the insertion mechanism preferably rotates and applies a distal force on cannula 200 and the applicator needle within cannula 200, such that cannula 200 is inserted through the wearer's skin. Cannula 200 may include a proximal cannula head configured to couple to one or more locking mechanisms on the pad skeleton and, at the same time, uncouple applicator 100 from the pad skeleton. The insertion mechanism further may be configured to continue rotating to withdraw the applicator needle from cannula 200 and to store the applicator needle within the applicator after cannula 200 is inserted.

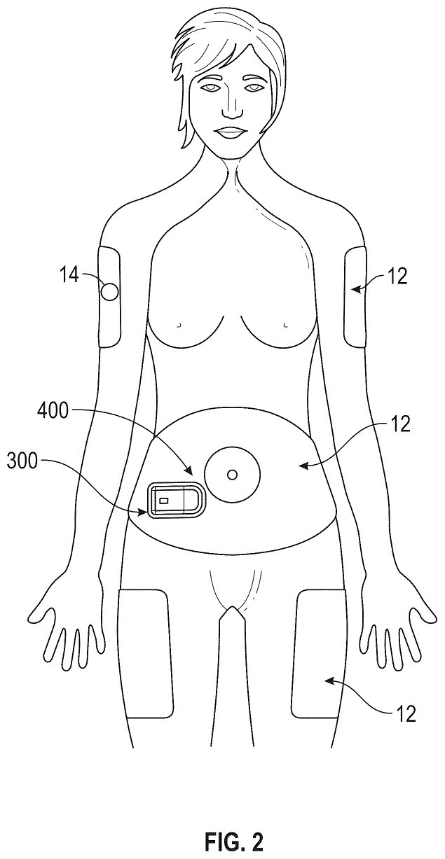

Referring now to FIG. 2, exemplary attachment zones for the patch pump and an optional external sensor, such as a continuous glucose monitoring sensor are illustrated. Attachment zones 12 illustrate several locations on the wearer's body where the applicator may attach the adhesive pad and insert the cannula and to which the patch pump is secured. For example, the patch pump may be secured to the upper arms, abdomen, or thighs of the wearer. As will also be understood by one of ordinary skill in the art, the patch pump may be secured to other locations on the wearer.

The patch pump also may be operatively coupled to an optional continuous glucose monitoring sensor, which may transmit data to a controller of the patch pump, which data may be used to adjust the time of insulin delivery or the amount of each dose. Preferably, the patch pump receives data from continuous glucose monitoring sensor 14, which is configured to be attached within attachment zones 12.

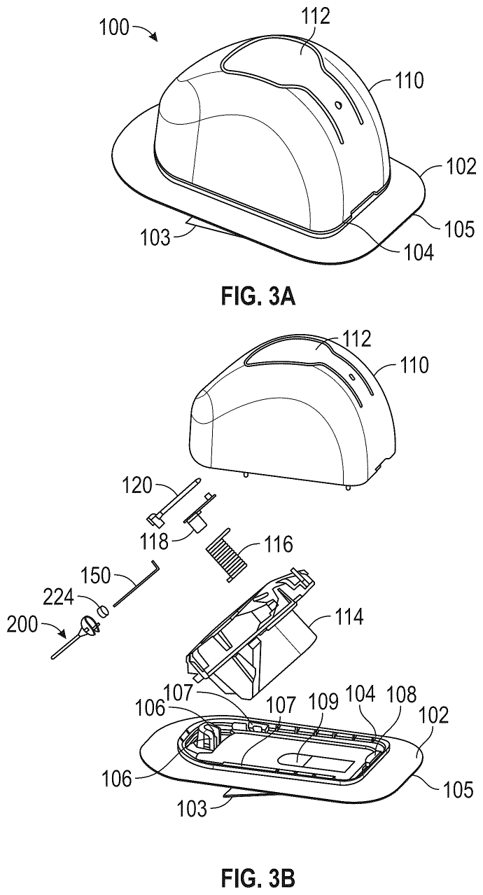

Referring now to FIGS. 3A and 3B, perspective and exploded views of an exemplary pad and applicator are described. Applicator 100 may transcutaneously apply a cannula, upon actuation by a user, which is designed to deliver doses of medication (e.g., insulin) from a patch pump configured to be removable coupled to the cannula. Advantageously, applicator further may apply a pad that is adhered to the wearer's skin and then coupled to the patch pump. For example, actuation of applicator 100 may both insert the cannula and cause the cannula to be locked to the adhesive pad in a single actuation. Further, applicator 100 may include internal components designed to minimize noise during the actuation process. For example, the applicator 100 may avoid clicks and/or hard stops that make audible noises during insertion of the cannula.

In a pre-actuation state, applicator 100 may be coupled to pad 102 as shown in FIG. 3A. For example, applicator 100 may be coupled to pad 102 via pad skeleton 104 of pad, which is disposed on a first surface of pad 102. Skin-safe pad adhesive 105 may be disposed on a second, skin-facing surface of pad 102 such that the pump-pad assembly may be attached to a wearer for a period of time, for example, 3-5 days, 3-10 days, or 10 days or more. One or more release liners 103 may be attached to pad adhesive 105 until pad 102 is ready to be secured to the wearer. Pad skeleton 104 may be a frame with a shape designed to surround the pump-cap assembly so as to securely couple the adhesive pad to the pump-cap for wearing by the patient. Pad skeleton 104 may be designed to removably couple portions of pad 102 to applicator 100 in the pre-actuation state. For example, pad skeleton 104 may have one or more attachment mechanisms to lock pad 102 to applicator 100 and unlock upon actuation of applicator 100. Advantageously, the attachment mechanisms also may lock the cannula to pad 102 after actuation. As depicted in FIG. 3A, pad skeleton 104 may have pad attachments 106 at a first end of pad 102 and pad back clip 108 at a second end of pad 102. Pad attachments 106 and pad back clip 108 may interact with applicator 100 or a patch pump to lock the pad to applicator 100 or the patch pump. Pad attachments 106 may include at least two arms that protrude upwards from the pad and away from the skin surface of the wearer. Each arm may have an opening (e.g., slot) to receive extensions from the applicator during pre-actuation and extensions from the cannula post-actuation. Thus, the arms, which may have a U-shape, and openings may be used to lock to both the applicator and the cannula. Pad skeleton 104 may also include pad clips holes 107 disposed on the sides of pad skeleton 104. Pad clips holes 107 may be a hole or receptacle sized and shaped to interact with a corresponding feature of the pump-cap assembly such that the pump-cap assembly may be locked to the pad. Further, pad 102 may include pad opening 109 to allow direct sensing of the wearer's skin by one or more sensors of the pump. For example, the skin sensor(s) and/or the PPG sensor(s) may be positioned at pad opening 109 when the pump is coupled to the pad.

Applicator 100 may include applicator housing 110 and actuator 112. Applicator housing 110 is configured to house the mechanisms for inserting the cannula. After insertion of the cannula, internal component 114 is designed to withdraw and safely store the needle used to pierce the wearer's skin. Actuator 112, upon actuation, causes the cannula to be transcutaneously inserted into the wearer's skin. Actuation of actuator 112 also may unlock applicator 100 from pad 102. Actuation of actuator 112 also may lock the transcutaneously inserted cannula into pad 102. For example, actuation of applicator 100 may insert the cannula transcutaneously, unlock the applicator from the pad, and lock the cannula to the pad in a single actuation. Actuator 112 may release the internal mechanism disposed within applicator housing 110 when actuated by the wearer, thus causing the cannula to advance through the wearer's skin. Actuator 112 may be a button configured to be pressed by the wearer as illustrated, or may be a lever, snap, knob, or the like. The mechanism for inserting the cannula may include internal component 114, biasing member 1156, and links 118 and 120, which are disposed within applicator housing 110, and are configured to advance cannula 200 through pad 102 and into the wearer's skin. The mechanism may further include applicator needle 150, which is configured to be disposed within cannula 200 during insertion and withdrawn from cannula 200 after insertion. Self-sealing septum 224 may be disposed within the cannula head of cannula 200 in order to support and guide applicator needle 150 and minimize backflow out of cannula 200.

As set forth above, upon actuator of the actuator, the pad 102 is released from the applicator 100 and the cannula 200 advanced through the pad 102. This is an irreversible process such that the applicator 100 is a single use device that cannot be actuated a second time. If the applicator 100 is accidentally actuated, the pad 102 and cannula 200 will need to be discarded without being used and the user will have to use a new applicator 100. As such, it is critical that the applicator 100 not be actuated until properly positioned on the user.

Prior to use, an applicator may be contained in a blister pack, which generally includes a clear thin plastic case surrounding and generally conforming to a shape of the applicator attached to a sheet of cardboard on which the applicator rests. Because the actuator 112 of applicator 100 is on an upper, external surface of the applicator, in such a packaging configuration the actuator 112 is covered by only the thin layer of plastic of the plastic case. As such, it would take minimal force on the plastic case surrounding the applicator 100 in the area of the actuator to inadvertently actuate the applicator 100. This could occur, for example, during shipping of a large number of applicators together in a common package, during storage of an applicator if the applicator is moved or contacted by other items, when a user is transporting a sealed applicator in a backpack, purse, when a user is opening the packaging, etc. and in a number of other circumstances.

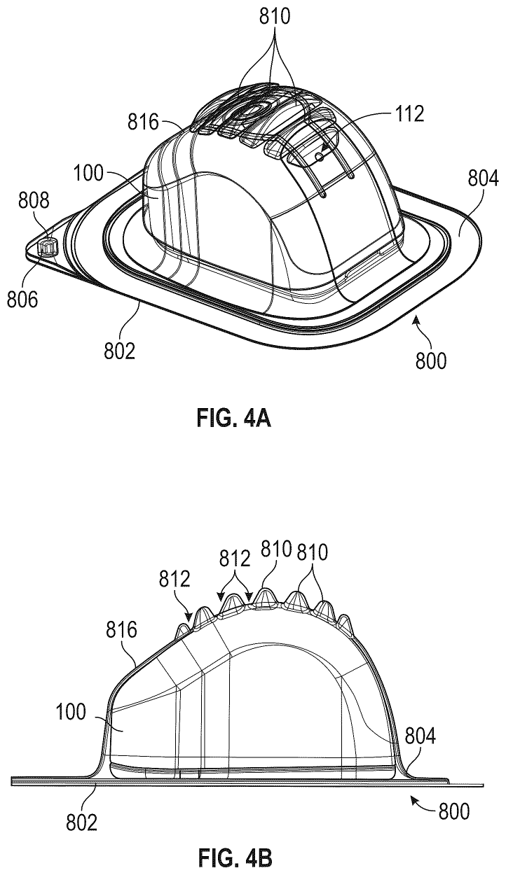

As such, the present application provides for packaging for an applicator such as applicator 100 disclosed herein that reduces the possibility of inadvertent actuation. One example of such packaging 800 containing an applicator 100 is depicted in FIGS. 4A-4D. Packing 800 is configured as a blister pack having a cardboard base 802 on which the applicator 100 rests with a plastic case 804 releasably attached to the cardboard base 802 and extending around and generally conforming to a shape of applicator 100. The cardboard base 802 may include one or more projections 806 and plastic case 804 may include corresponding recesses 808 that provide a press fit with projections to releasably retain the plastic case 804 on the cardboard base 802.

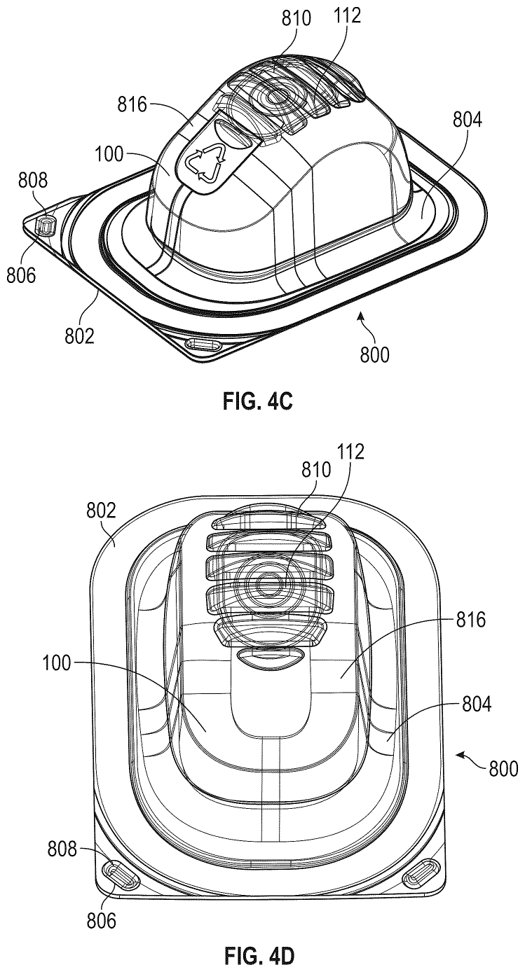

To prevent inadvertent actuation of the applicator 100 due to forces being transmitted through plastic case 804 onto actuator 112 of applicator 100, plastic case 804 is formed with a plurality of ridges 810 spaced apart by recesses 812 along the area of plastic case 804 over actuator 112. As can be seen most clearly in the partial view of FIG. 5, each ridge 810 includes a pair of opposing sidewalls 814 extending upwardly towards each other from a body portion 816 of plastic case 804 that conforms to a shape of applicator 100 that meet at a tapered tip 818. A bottom surface of each recess 812 may therefore be directly adjacent the outer surface of applicator. Each ridge 810 is therefore wider at a base of the ridge at the body portion 816 of the plastic case than at the tapered tip 818 of the ridge 810. These ridges 810 require significantly more force to be deformed to transmit force onto actuator 112 of applicator 100 than a smooth, continuous plastic case conforming to a shape of applicator in the region of actuator. In particular, ridges 810 are designed such that typical forces on plastic case 804 during shipping and storage as well as during handling by a user to remove an applicator from packaging 800 will not be transmitted onto actuator 112. It should be noted that the depicted ridges 810 are just one example of features that reduce the transmission of forces on the plastic case 804 onto the actuator and that various other shapes, orientations, features, etc. can alternatively be provided on plastic case to accomplish the function. For example, while ridges 810 are depicted having a generally rounded tapered tip 818, ridges could employ any other configuration such as a pointed tip, square tip, etc.

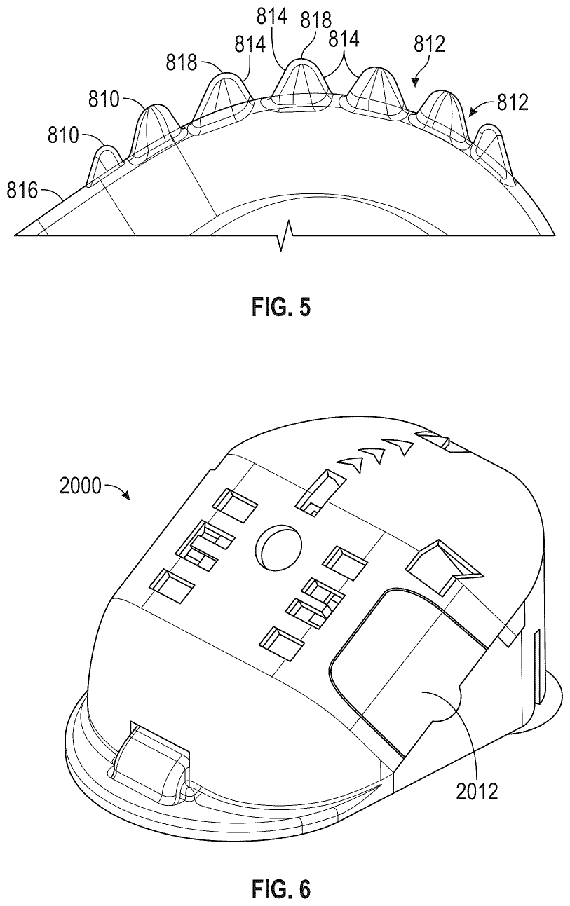

As such, use of such packaging 800 significantly reduces the risk of inadvertent activation of the actuator 112 of such an applicator 100. It should be noted that such a packaging concept can be applied to applicators having other designs. For example, FIG. 6 depicts an applicator 2000 having a pair of actuators 2012 on opposing sides of applicator 2000 that are simultaneously depressed to actuate the applicator 2000. For such a design, ridges or similar features can be provided along the opposing sides of the packaging over the actuators 2012 to reduce the possibility of inadvertent actuation. In addition, packaging disclosed herein can be adapted to other medical devices as well as non-medical devices that include external actuation mechanisms to reduce the transmission of force from outside of the packaging onto those mechanisms.

In embodiments, a package configured to contain a medical device configured to be actuated by an external actuation mechanism on the medical device can include a generally planar base and a flexible case selectively attachable to the generally planar base to selectively enclose the medical device between the generally planar base and the flexible case. The flexible case can include a body portion generally conforming to a shape of the medical device and a plurality of ridges extending upwardly from the body portion over the external actuation mechanism when the medical device is enclosed between the generally planar base and the flexible case.

In some embodiments, each of the plurality of ridges comprises a pair of opposing sidewalls extending upwardly from the body portion, the body portion defining a plurality of recesses between each pair of the plurality of ridges.

In some embodiments, the pair of sidewalls of each of the plurality of ridges meet at a tapered tip.

In some embodiments, the tapered tip of each of the plurality of ridges is generally rounded.

In some embodiments, each of the plurality of ridges is wider at a base adjacent the body portion than at a tip of each of the plurality of ridges.

In some embodiments, the flexible case comprises a plastic material.

In some embodiments, the plurality of ridges is a first plurality of ridges configured to be disposed over a first actuation mechanism of the medical device and further comprising a second plurality of ridges configured to be disposed over a second actuation mechanism of the medical device.

In some embodiments, the medical device is a single use device configured to be disposed of following actuation of the external actuation mechanism

[symbol]

In some embodiments, the external actuation mechanism is one or more buttons on an outer housing of the medical device.

In some embodiments, the medical device is an applicator that aids in attaching a user-wearable infusion pump to a user.

In embodiments, a medical device system can include a medical device configured to be actuated by an external actuation mechanism on the medical device and a package including a generally planar base and a flexible case selectively attachable to the generally planar base to selectively enclose the medical device between the generally planar base and the flexible case. The flexible case can include a body portion generally conforming to a shape of the medical device and a plurality of ridges extending upwardly from the body portion over the external actuation mechanism when the medical device is enclosed between the generally planar base and the flexible case.

In some embodiments, each of the plurality of ridges comprises a pair of opposing sidewalls extending upwardly from the body portion, the body portion defining a plurality of recesses between each pair of the plurality of ridges.

In some embodiments, the pair of sidewalls of each of the plurality of ridges meet at a tapered tip.

In some embodiments, the tapered tip of each of the plurality of ridges is generally rounded.

In some embodiments, each of the plurality of ridges is wider at a base adjacent the body portion than at a tip of each of the plurality of ridges.

In some embodiments, the flexible case comprises a plastic material.

In some embodiments, the plurality of ridges is a first plurality of ridges and the actuation mechanism is a first actuation mechanism and wherein the medical device further comprises a second actuation mechanism and the flexible case further comprises a second plurality of ridges configured to be disposed over the second actuation mechanism.

In some embodiments, the medical device is a single use device configured to be disposed of following actuation of the external actuation mechanism

[symbol]

In some embodiments, the external actuation mechanism is one or more buttons on an outer housing of the medical device.

In some embodiments, the medical device is an applicator that aids in attaching a user-wearable infusion pump to a user.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

1. A package configured to contain a medical device configured to be actuated by an external actuation mechanism on the medical device, comprising:
a generally planar base; and
a flexible case selectively attachable to the generally planar base to selectively enclose the medical device between the generally planar base and the flexible case, wherein the flexible case includes a body portion generally conforming to a shape of the medical device and a plurality of ridges extending upwardly from the body portion over the external actuation mechanism when the medical device is enclosed between the generally planar base and the flexible case.

2. The package of claim 1, wherein each of the plurality of ridges comprises a pair of opposing sidewalls extending upwardly from the body portion, the body portion defining a plurality of recesses between each pair of the plurality of ridges.

3. The package of claim 2, wherein the pair of sidewalls of each of the plurality of ridges meet at a tapered tip.

4. The package of claim 3, wherein the tapered tip of each of the plurality of ridges is generally rounded.

5. The package of claim 1, wherein each of the plurality of ridges is wider at a base adjacent the body portion than at a tip of each of the plurality of ridges.

6. The package of claim 1, wherein the flexible case comprises a plastic material.

7. The package of claim 1, wherein the plurality of ridges is a first plurality of ridges configured to be disposed over a first actuation mechanism of the medical device and further comprising a second plurality of ridges configured to be disposed over a second actuation mechanism of the medical device.

8. The package of claim 1, wherein the medical device is a single use device configured to be disposed of following actuation of the external actuation mechanism.

9. The package of claim 1, wherein the external actuation mechanism is one or more buttons on an outer housing of the medical device.

10. The package of claim 1, wherein the medical device is an applicator that aids in attaching a user-wearable infusion pump to a user.

11. A medical device system comprising:
a medical device configured to be actuated by an external actuation mechanism on the medical device; and
a package comprising a generally planar base and a flexible case selectively attachable to the generally planar base to selectively enclose the medical device between the generally planar base and the flexible case, wherein the flexible case includes a body portion generally conforming to a shape of the medical device and a plurality of ridges extending upwardly from the body portion over the external actuation mechanism when the medical device is enclosed between the generally planar base and the flexible case.

12. The system of claim 11, wherein each of the plurality of ridges comprises a pair of opposing sidewalls extending upwardly from the body portion, the body portion defining a plurality of recesses between each pair of the plurality of ridges.

13. The system of claim 12, wherein the pair of sidewalls of each of the plurality of ridges meet at a tapered tip.

14. The system of claim 13, wherein the tapered tip of each of the plurality of ridges is generally rounded.

15. The system of claim 11, wherein each of the plurality of ridges is wider at a base adjacent the body portion than at a tip of each of the plurality of ridges.

16. The system of claim 11, wherein the flexible case comprises a plastic material.

17. The system of claim 11, wherein the plurality of ridges is a first plurality of ridges and the actuation mechanism is a first actuation mechanism and wherein the medical device further comprises a second actuation mechanism and the flexible case further comprises a second plurality of ridges configured to be disposed over the second actuation mechanism.

18. The system of claim 11, wherein the medical device is a single use device configured to be disposed of following actuation of the external actuation mechanism.

19. The system of claim 11, wherein the external actuation mechanism is one or more buttons on an outer housing of the medical device.

20. The package of claim 11, wherein the medical device is an applicator that aids in attaching a user-wearable infusion pump to a user.
